(19) United States
(12) Patent Application Publication
(10) Pub. No.: US 2011/0014148 A1
(43) Pub. Date: Jan. 20, 2011

(54) Cationic Surfactants Having Improved Properties

(75) Inventors: Ansgar Behler, Bottrop (DE); Frank Clasen, Hilden (DE); Hans-Martin Haake, Erkrath (DE); Monika Barbenheim, Bottrop (DE)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Dusseldorf (DE)

(21) Appl. No.: 12/922,239

(22) PCT Filed: Aug. 16, 2008
(86) PCT No.: PCT/EP08/06751

§ 371 Date: Sep. 13, 2010

(30) Foreign Application Priority Data
Mar. 12, 2008 (EP) 08004512

(51) Int. Cl. A61K 8/41; C07C 69/24; A61Q 5/12

(52) U.S. Cl. 424/70.28; 560/253

(57) ABSTRACT

The invention is directed to cationic surfactants of formula (I), where R1 is a saturated or unsaturated, branched or linear alkyl group having 6 to 22 carbon atoms, R2 and R3 independently are C1-C3 alkyl groups or hydrogen, n is 2 or 3, and X⁻ is an anion selected from the group consisting of halides, deprotonated carboxylic acids and methosulfate. The cationic surfactants are useful, particularly as hair conditioners, for the preparation of cosmetic compositions.

### CROSS-REFERENCE TO RELATED APPLICATIONS

This application is the US national stage entry under USC 35 §371 of international application no. PCT/EP2008/006751, filed on Aug. 16, 2008, which claims priority to European patent application no. 08004512, filed on Mar. 12, 2008, both of which are incorporated herein in their entireties.

### FIELD OF THE INVENTION

The invention is in the field of cationic surfactants and cosmetic preparations which comprise these surfactants.

### BACKGROUND OF THE INVENTION

An important aspect in hair care is the conditioning of the hair, e.g. after washing, since it is exposed daily to a variety of influencing factors such as sunlight, environmental grime or chlorinated water, but also various treatments such as washing, bleaching, coloring, waving, combing or blow-drying. All this can lead, alone or in combination, to hair damage which can manifest itself in the hair surface becoming rougher. Moreover, in the case of damaged hair, hydrophilization of the surface, an increase in the negative charges on the hair and reduced mechanical stability are observed. This can lead to the hair being combable only with difficulty, quickly breaking and having split ends. The feel changes detrimentally and the natural shine of the hair is lost.

Consequently, conditioners, which improve the combability, are used in cosmetic hair treatment compositions. Electrostatic charging of the hair is prevented or minimized, the hair shines again and its surface is again significantly more hydrophobic. Furthermore, the mechanical strength of hair is increased as a result of conditioners, even during otherwise harmful chemical treatments.

Many different substance classes have already been used for this purpose in cosmetic preparations. By way of example, mention may be made here of consistency regulators or lipophilic conditioners such as fatty alcohols or partial glycerides. Silicones are used as secondary conditioners. Conditioning properties are also attributed to emulsifiers such as fatty alcohol ethoxylates, alkyl polyglycosides and polymers, as well as wetting agents and thickeners.

Furthermore, cationic surfactants may also be mentioned as a conditioning component in cosmetic preparations. These are mostly alkyl ammonium compounds with at least one long alkyl chain in the molecule. The counterion present is often a halide, such as chloride, or methosulfate. Examples of such cationic surfactants are cetrimonium chloride, distearoyl hydroxyethylmonium methosulfate, distearyldimonium chloride or behentrimonium chloride.

All of these surfactants have exceptional conditioning properties, although they do also have a number of disadvantages. They generally have poor biodegradability, and some also have high aquatic toxicity and are not very skin-friendly.

The object of the present patent application was therefore to provide novel cationic conditioners which do not have the aforementioned disadvantages, but nevertheless have exceptional conditioning properties.

### DETAILED DESCRIPTION OF PREFERRED EMBODIMENTS

The present patent application therefore provides cationic surfactants of the formula (I)

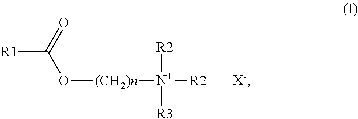

in which R1 is a saturated or unsaturated, branched or linear alkyl radical having 6 to 22 carbon atoms, R2 and R3, independently of one another, are alkyl radicals having 1 to 3 carbon atoms or hydrogen, n is 2 or 3 and X is halogens, deprotonated carboxylic acids or methosulfate.

In one preferred embodiment, use is made of surfactants according to formula (I) in which R1 is a saturated or unsaturated, branched or linear alkyl radical having 12 to 22 carbon atoms, R2 is methyl and R3 is methyl or hydrogen, n is 2 and X is chlorine. The surfactants of the formula (I) having a relatively long alkyl chain R1 are characterized by better conditioning properties, as demonstrated in the examples.

In this connection, very particular preference is given to the cationic surfactants of the formula (I) when R1 is a saturated or unsaturated, branched or linear alkyl radical having 18 to 22 carbon atoms.

The aforementioned cationic surfactants are used for producing cosmetic preparations. They are used as conditioners in these preparations. The use amounts in these preparations vary here in quantitative ratios of from 0.1 to 20% by weight, preferably from 0.1 to 10% by weight and particularly preferably from 0.5 to 5% by weight, based on the total formulation.

Suitable deprotonated carboxylic acids specified as radical X are in particular organic acids such as citric acid, lactic acid or acetic acid.

### INDUSTRIAL APPLICABILITY

The present invention further provides cosmetic preparations which comprise at least one cationic surfactant according to formula (I). The cosmetic preparations may be a wide variety of surfactant-based preparations, such as, for example, shampoos, conditioners, shower baths, shower gels, foam baths, oil baths, 2-in-1 shampoos, etc.

Furthermore, the cationic surfactants of the formula (I) and according to the invention can be used in detergents and cleaners, e.g. in cleaners for the cleaning of hard surfaces or in dishwashing compositions, either for manual or machine washing.

As further auxiliaries and additives, these compositions can comprise mild surfactants, oil bodies, emulsifiers, pearlescent waxes, superfatting agents, stabilizers, polymers, silicone compounds, fats, waxes, lecithins, phospholipids, biogenic active ingredients, UV sun protection factors, antioxidants, deodorants, antiperspirants, antidandruff agents, film formers, swelling agents, insect repellants, self-tanning agents, tyrosine inhibitors (depigmentation agents), hydrotropes, solubilizers, preservatives, perfume oils, dyes and the like.

### Surfactants

As surface-active substances, anionic, nonionic, cationic and/or amphoteric or zwitterionic surfactants may be present, the fraction of which in the compositions is usually about 1 to 70, preferably 5 to 50 and in particular 10 to 30% by weight. Typical examples of anionic surfactants are soaps, alkyl-benzenesulfonates, alkanesulfonates, olefinsulfonates, alkyl ether sulfonates, glyceryl ether sulfonates, α-methyl ester sulfonates, sulfofatty acids, alkyl sulfates, fatty alcohol ether sulfates, glyceryl ether sulfates, fatty acid ether sulfates, hydroxy mixed ether sulfates, monoglyceride (ether) sulfates, fatty acid amide (ether) sulfates, mono- and alkoxylated and nonalkoxylated dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, alkyl oligo-glucoside carboxylates, protein fatty acid condensates (in particular wheat-based vegetable products) and alkyl (ether) phosphates. If the anionic surfactants contain polyglycol ether chains, these can have a conventional homolog distribution, but preferably have a narrowed homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formulas, optionally partially oxidized alk(en)yl oligoglycosides or glucoronic acid derivatives, fatty acid N-alkyl-glucamides, protein hydrolyzates (in particular wheat-based vegetable products), polyol fatty acid esters, sugar esters, sorbitan esters, polysorbates and amine oxides. If the nonionic surfactants contain polyglycol ether chains, these can have a conventional homolog distribution, but preferably have a narrowed homolog distribution. Typical examples of cationic surfactants are quaternary ammonium compounds, such as, for example, dimethyldistearylammonium chloride or cetyl-trimonium chloride, and ester quats, in particular quaternized fatty acid trialkanolamine ester salts.

Typical examples of amphoteric or zwitterionic surfactants are alkylbetaines, alkylamidobetaines, aminopropionates, aminoglycinates, imidazolinium-betaines and sulfobetaines. The specified surfactants are exclusively known compounds. Typical examples of particularly suitable mild, i.e. particularly skin-friendly, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkyl-amidobetaines, amphoacetals and/or protein fatty acid condensates, the latter preferably based on wheat proteins.

### Oil Bodies

Oil bodies are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, such as e.g. myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, ercyl myristate, erucyl palmitate, erucyl stearate, erucyl isostearate, erucyl oleate, erucyl behenate and erucyl erucate. Also suitable in consideration are esters of linear C₆-C₂₂-fatty acids with branched alcohols, in particular 2-ethylhexanol, esters of C₁₈-C₃₈-alkylhydroxycarboxylic acids with linear or branched C₆-C₂₂-fatty alcohols, in particular dioctyl malate, esters of linear and/or branched fatty acids with polyhydric alcohols (such as e.g. propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as e.g. dicaprylyl carbonate (CETIOL® CC), Guerbet carbonates based on fatty alcohols having 6 to 18, preferably 8 to 10, carbon atoms, esters of benzoic acid with linear and/or branched C₆-C₂₂-alcohols (e.g. FINSOLV® TN), linear or branched, symmetrical or asymmetrical dialkyl ethers having 6 to 22 carbon atoms per alkyl group, such as e.g. dicaprylyl ether (CETIOL® OE), ring-opening products of epoxidized fatty acid esters with polyols, silicone oils (cyclomethicones, silicon methicone types etc.) and/or aliphatic or naphthenic hydrocarbons, such as e.g. squalane, squalene or dialkylcyclohexanes.

### Emulsifiers

Suitable emulsifiers are, for example, nonionic surfactants from at least one of the following groups:
addition products of from 2 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols, having 8 to 15 carbon atoms in the alkyl group, and alkylamines having 8 to 22 carbon atoms in the alkyl radical;
alkyl and/or alkenyl oligoglycosides having 8 to 22 carbon atoms in the alk(en)yl radical and ethoxylated analogs thereof;
addition products of from 1 to 15 mol of ethylene oxide onto castor oil and/or hydrogenated castor oil;
addition products of from 15 to 60 mol of ethylene oxide onto castor oil and/or hydrogenated castor oil;
partial esters of glycerol and/or sorbitan with unsaturated, linear or saturated, branched fatty acids having 12 to 22 carbon atoms and/or hydroxy-carboxylic acids having 3 to 18 carbon atoms, and also adducts thereof having 1 to 30 mol of ethylene oxide;
partial esters of polyglycerol (average degree of self-condensation 2 to 8), polyethylene glycol (molecular weight 400 to 5000), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear and/or branched fatty acids having 12 to 22 carbon atoms and/or hydroxycarboxylic acids having 3 to 18 carbon atoms, and adducts thereof with 1 to 30 mol of ethylene oxide;
mixed esters of pentaerythritol, fatty acids, citric acid and fatty alcohol and/or mixed esters of fatty acids having 6 to 22 carbon atoms, methyl glucose and polyols, preferably glycerol or polyglycerol.
   mono-, di- and trialkyl phosphates, and mono-, di- and/or tri-PEG alkyl phosphates and salts thereof;
   wool wax alcohols;
   polysiloxane-polyalkyl-polyether copolymers and corresponding derivatives;
   block copolymers, e.g. polyethylene glycol-30 dipolyhydroxystearate;
   polymer emulsifiers, e.g. PEMULEN® grades (TR-1, TR-2) from Goodrich;
   polyalkylene glycols and)
   glycerol carbonate.

Ethylene Oxide Addition Products

The addition products of ethylene oxide and/or of propylene oxide onto fatty alcohols, fatty acids, alkylphenols or onto castor oil are known, commercially available products. These are homolog mixtures, the average degree of alkoxylation of which corresponds to the ratio of the quantitative amounts of ethylene oxide and/or propylene oxide and substrate with which the addition reaction is carried out. C_{12/18}-fatty acid mono- and diesters of addition products of ethylene oxide onto glycerol are known as refatting agents for cosmetic preparations.

Alkyl and/or Alkenyl Oligoglycosides

Alkyl and/or alkenyl oligoglycosides, their preparation and their use are known from the prior art. Their preparation takes place in particular by reacting glucose or oligosaccharides with primary alcohols having 8 to 18 carbon atoms. As regards the glycoside radical, either monoglycosides in which one cyclic sugar radical is glycosidically bonded to the fatty alcohol, or oligomeric glycosides with a degree of oligomerization up to preferably about 8 are suitable. The degree of oligomerization here is a statistical average value based on a homolog distribution customary for such technical-grade products.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid moglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric diglyceride, malic acid monoglyceride, malic acid diglyceride, and technical-grade mixtures thereof, which may also comprise small amounts of triglyceride in secondary amounts from the production process. Addition products of from 1 to 30, preferably 5 to 10, mol of ethylene oxide onto the specified partial glycerides are likewise suitable.

Sorbitan Esters

Sorbitan esters are sorbitan monoisostearate, sorbitan sesquiisostearate, sorbitan diisostearate, sorbitan triisostearate, sorbitan monooleate, sorbitan sesqui-oleate, sorbitan dioleate, sorbitan trioleate, sorbitan monoerucate, sorbitan sesquierucate, sorbitan dierucate, sorbitan trierucate, sorbitan monoricin-oleate, sorbitan sesquiricinoleate, sorbitan diricin-oleate, sorbitan triricinoleate, sorbitan monohydroxy-stearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and technical-grade mixtures thereof. Addition products of from 1 to 30, preferably 5 to 10, mol of ethylene oxide onto the specified sorbitan esters are likewise suitable.

Polyglyercol Esters

Typical examples of suitable polyglycerol esters are polyglycerol-2 dipolyhydroxystearate (DEHYMULS® PGPH), polyglycerol-3 diisostearate (LAMEFORM® TGI), polyglyceryl-4 isostearate (ISOLAN® GI 34), polyglyceryl-3 oleate, diisostearoyl polyglyceryl-3 diisostearate (ISOLAN® PDI), polyglyceryl-3 methyl-glucose distearate (TEGO CARE® 450), polyglyceryl-3 beeswax (CERA BELLINA®), polyglyceryl-4 caprate (Polyglycerol Caprate T2010/90), polyglyceryl-3 cetyl ether (CHIMEXANE® NL), polyglyceryl-3 distearate (CREMOPHOR® GS 32) and polyglyceryl polyricinoleate (ADMUL® WOL 1403) polyglyceryl dimerate isostearate, and mixtures thereof. Examples of further suitable polyol esters are the mono-, di- and triesters of trimethylolpropane or pentaerythritol with lauric acid, coconut fatty acid, tallow fatty acid, palmitic acid, stearic acid, oleic acid, behenic acid and the like, optionally reacted with 1 to 30 mol of ethylene oxide.

Anionic Emulsifiers

Typical anionic emulsifiers are aliphatic fatty acids having 12 to 22 carbon atoms, such as, for example, palmitic acid, stearic acid or behenic acid, and also dicarboxylic acids having 12 to 22 carbon atoms, such as, for example, azeleic acid or sebacic acid.

Amphoteric and Cationic Emulsifiers

Furthermore, zwitterionic surfactants can be used as emulsifiers. Zwitterionic surfactants is the term used to refer to those surface-active compounds which carry in the molecule at least one quaternary ammonium group and at least one carboxylate group or one sulfonate group. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethyl hydroxyethylcarboxymethyl glycinate. Particular preference is given to the fatty acid amide derivative known under the CTFA name Cocamidopropyl Betaine. Likewise suitable emulsifiers are ampholytic surfactants. Ampholytic surfactants are understood as meaning those surface-active compounds which, apart from a C_{8/18}-alkyl or acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylamino-propionate and C_{12/18}-acylsarcosine. Finally, cationic surfactants are suitable as emulsifiers, particular preference being given to those of the esterquat type, preferably methyl-quaternized difatty acid triethanol-amine ester salts.

### Fats and Waxes

Typical examples of fats are glycerides, i.e. solid or liquid vegetable or animal products which consist essentially of mixed glycerol esters of higher fatty acids, suitable waxes are inter alia natural waxes, such as e.g. candelilla wax, carnauba wax, Japan wax, espartograss wax, cork wax, guaruma wax, rice germ oil wax, sugar cane wax, ouricury wax, montan wax, beeswax, shellac wax, spermaceti, lanolin (wool wax), uropygial grease, ceresin, ozokerite (earth wax), petrolatum, paraffin waxes, microwaxes; chemically modified waxes (hard waxes), such as e.g. montan ester waxes, sasol waxes, hydrogenated jojoba waxes, and synthetic waxes, such as e.g. polyalkylene waxes and polyethylene glycol waxes. Besides the fats, suitable additives are also fat-like substances, such as lecithins and phospholipids. The term lecithins is understood by the person skilled in the art as meaning those glycerol-phospholipids which are formed from fatty acids, glycerol, phosphoric acid and choline as a result of esterification. In the specialist world, lecithins are therefore also often as phosphatidylcholines (PC). Examples of natural lecithins which may be mentioned are the cephalins, which are also referred to as phosphatidic acids and are derivatives of 1,2-diacyl-sn-glyercyl-3-phosphoric acids. By contrast, phospho-lipids are usually understood as meaning mono- and preferably diesters of phosphoric acid with glycerol (glycerol phosphates), which are generally classed as fats. In addition, sphingosines and sphingolipids are also suitable.

### Pearlescent Waxes

Suitable pearlescent waxes are, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which in total have at least 24 carbon atoms, specifically laurone and distearyl ether; fatty acids such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxy groups, and mixtures thereof.

### Superfatting Agents

Superfatting agents which can be used are substances such as, for example, lanolin and lecithin, and polyethoxylated or acylated lanolin and lecithin derivatives, polyol fatty acid esters, monoglycerides and fatty acid alkanolamides, the latter simultaneously serving as foam stabilizers.

### Stabilizers

Stabilizers which can be used are metal salts of fatty acids, such as e.g. magnesium stearate or ricinoleate, aluminum stearate or ricinoleate and/or zinc stearate or ricinoleate.

### Polymers

Suitable cationic polymers are, for example, cationic cellulose derivatives, such as, for example, a quaternized hydroxyethyl cellulose which is available under the name POLYMER JR 400® from Amerchol, cationic starch, copolymers of diallyl ammonium salts and acrylamides, quaternized vinylpyrrolidone/vinylimidazole polymers, such as, for example, LUVIQUAT® (BASF), condensation products of polyglycols and amines, quaternized collagen polypeptides, such as, for example, lauryldimonium hydroxypropyl hydrolyzed collagen (LAMEQUAT® L/Grünau), quaternized wheat polypeptides, polyethyleneimine, cationic silicone polymers, such as, for example, amodimethicones, copolymers of adipic acid and dimethylaminohydroxy-propyldiethylenetriamine (CARTARETINE®/Sandoz), copolymers of acrylic acid with dimethyldiallyl-ammonium chloride (MERQUAT® 550/Chemviron), polyamino-polyamides, and crosslinked water-soluble polymers thereof, cationic chitin derivatives, such as, for example, quaternized chitosan, optionally in micro-crystalline distribution, condensation products of dihaloalkylene, such as, for example, dibromobutane with bisdialkylamines, such as, for example, bis-dimethylamino-1,3-propane, cationic guar gum, such as, for example, JAGUAR® CBS, JAGUAR® C-17, JAGUAR® C-16 from Celanese, quaternized ammonium salt polymers, such as, for example MIRAPOL® A-15, MIRAPOL® AD-1, MIRAPOL® AZ-1 from Miranol.

Suitable anionic, zwitterionic, amphoteric and nonionic polymers are, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyltrimethylammonium chloride/acrylate copolymers, octylacrylamide/methyl methacrylate/tert-butylaminoethyl methacrylate/2-hydroxypropyl methacrylate copolymers, polyvinyl-pyrrolidone, vinylpyrrolidone/vinyl acetate copolymers, vinylpyrrolidone/dimethylaminoethyl methacrylate/vinyl-caprolactam terpolymers, and optionally derivatized cellulose ethers and silicones.

### Silicone Compounds

Suitable silicone compounds are, for example, dimethyl-polysiloxanes, methylphenylpolysiloxanes, cyclic silicones, and also amino-, fatty-acid-, alcohol-, polyether-, epoxy-, fluorine-, glycoside- and/or alkyl-modified silicone compounds which, at room temperature, may be present either in liquid form or else in resin form. Also of suitability are simethicones, which are mixtures of dimethicones having an average chain length of from 200 to 300 dimethylsiloxane units and hydrogenated silicates.

### Biogenic Active Ingredients

Biogenic active ingredients are to be understood as meaning, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, such as e.g. prune extract, bambara nut extract and vitamin complexes.

### Film Formers

Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid and salts thereof and similar compounds.

### Antidandruff Active Ingredients

Suitable antidandruff active ingredients are piroctone olamine (1-hydroxy-4-methyl-6-(2,4,4-trimethylpentyl)-2-(1H)-pyridinone monoethanolamine salt), BAYPIVAL® (climbazole), KETOCONAZOL®, (4-acetyl-1-{-4-[2-(2,4-dichlorophenyl)r-2-(1H-imidazol-1-ylmethyl)-1,3-dioxylan-c-4-ylmethoxyphenyl]piperazine, ketoconazole, elubiol, selenium disulfide, colloidal sulfur, sulfur polyethylene glycol sorbitan monooleate, sulfur ricinol polyethoxylate, sulfur tar distillates, salicylic acid (or in combination with hexachlorophene), undexylenic acid monoethanolamide sulfosuccinate Na salt, LAMEPON® UD (protein undecylenic acid condensate), zinc pyrithione, aluminum pyrithione and magnesium pyrithione/dipyrithione magnesium sulfate.

### Swelling Agents

Swelling agents for aqueous phases that can be used are montmorillonites, clay mineral substances, PEMULEN® and also alkyl-modified CARBOPOL® grades (Goodrich).

### Hydrotropes

To improve the flow behavior, it is also possible to use hydrotropes, such as, for example, ethanol, isopropyl alcohol, or polyols. Polyols which are suitable here preferably have 2 to 15 carbon atoms and at least two hydroxyl groups. The polyols can also contain further functional groups, in particular amino groups, and/or be modified with nitrogen. Typical examples are
glycerol;
alkylene glycols, such as, for example, ethylene glycol, diethylene glycol, propylene glycol, butylene glycol, hexylene glycol, and also polyethylene glycols having an average molecular weight of from 100 to 1000 Daltons;
technical-grade oligoglycerol mixtures with a degree of self-condensation of from 1.5 to 10, such as, for example, technical-grade diglycerol mixtures with a diglyercol content of from 40 to 50% by weight;
methylol compounds, such as in particular trimethylolethane, trimethylolpropane, trimethylol-butane, pentaerythritol and dipentaerythritol;
low alkyl glucosides, in particular those having 1 to 8 carbon atoms in the alkyl radical, such as, for example, methyl glucoside and butyl glucoside;
sugar alcohols having 5 to 12 carbon atoms, such as, for example, sorbitol or mannitol,
sugars having 5 to 12 carbon atoms, such as, for example, glucose or sucrose;
amino sugars, such as, for example, glucamine;
dialcohol amines, such as diethanolamine or 2-amino-1,3-propanediol.

### Preservatives

Suitable preservatives are, for example, phenoxy-ethanol, formaldehyde solution, parabens, pentanediol or sorbic acid, and also the silver complexes known under the name SURFACINE® and the further substance classes listed in Annex 6, Part A and B of the Cosmetics Ordinance.

### Perfume Oils and Aromas

Perfume oils which may be mentioned are mixtures of natural and synthetic fragrances. Natural fragrances are extracts of flowers (lily, lavender, rose, jasmine, neroli, ylang-ylang), stems and leaves (geranium, patchouli, petitgrain), fruits (anise, coriander, caraway, juniper), fruit peels (bergamot, lemon, orange), roots (mace, angelica, celery, cardamom, costus, iris, calmus), woods (pine wood, sandalwood, guajak wood, cedar wood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Animal raw materials are also suitable, such as, for example, civet and castoreum. Typical synthetic fragrance compounds are products of the ester, ether, aldehyde, ketone, alcohol and hydrocarbon types. Fragrance compounds of the ester type are, for example, benzyl acetate, phenoxyethyl isobutyrate, p-tert-butyl cyclohexyl acetate, linalyl acetate, dimethylbenzyl carbinyl acetate, phenylethyl acetate, linalyl benzoate, benzyl formate, ethylmethyl phenyl glycinate, allyl cyclohexyl propionate, styrallyl propionate and benzyl salicylate. The ethers include, for example, benzyl ethyl ether; the aldehydes include, for example, the linear alkanals having 8 to 18 carbon atoms, citral, citronellal, citronellyloxyacetaldehyde, cyclamenaldehyde, hydroxycitronellal, lilial and bourgeonal; the ketones include, for example, the ionones, α-isomethylionone and methyl cedryl ketone; the alcohols include anethol, citronellol, eugenol, isoeugenol, geraniol, linalool, phenylethyl alcohol and terpineol; the hydrocarbons include primarily the terpenes and balsams. However, preference is given to using mixtures of different perfume compounds, which together produce a pleasant scent note. Also suitable as perfume oils are essential oils of relatively low volatility, which are mostly used as aroma components, for example sage oil, chamomile oil, clove oil, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzyl acetone, cyclamen aldehyde, linalool, boisambrene forte, ambroxan, indole, hedione, sandelice, citrus oil, mandarin oil, orange oil, allyl amyl glycolate, cyclovertal, lavandin oil, clary sage oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat, alone or in mixtures.

Suitable aromas are, for example, peppermint oil, spearmint oil, anise oil, star anise oil, caraway oil, eucalyptus oil, fennel oil, citrus oil, wintergreen oil, clove oil, menthol and the like.

### Dyes

Dyes which can be used are the substances approved and suitable for cosmetic purposes. Examples are cochineal red A (C.I. 16255), patent blue V (C.I. 42051), indigo tin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are usually used in concentrations of from 0.001 to 0.1% by weight, based on the total mixture.

### EXAMPLES

### Example 1

### General Procedure for the Preparation of a Compound according to formula (i) where R1=C17-alkyl, n=2, R2=R3=CH₃, X=Cl

stearic acid dimethylethanolamine ester is introduced into an autoclave suitable for quaternizations and flushed several times with nitrogen. It is heated to 90° C., and methyl chloride is metered in portions. When the addition is complete, afterstirring is carried out for a further four hours at 90° C.

### Mixture

400 g (1.09 mol) of stearic acid dimethyl-ethanolamine ester
400 g of C16/18 alcohol (LANETTE® O)
52.1 g (1.09 mol) of methyl chloride
   Result: solid, readily pelletable product; cationic surfactant content 49.7%.

### Example 2

### Determination of the Conditioning Properties

### Half-Side Test

Comparative half-side tests were carried out under standardized conditions on 10 female subjects in order to determine essential properties of shampoos.

### Composition Used (Data as Active Substance Content)

The cationic surfactants were formulated in the following composition:

2% by weight of cationic surfactant
3.2% by weight of C16/18 fatty alcohol
0.8% by weight of ceteareth-20
ad 100 water
pH 3.5-4.0 (pH adjustment with citric acid)

TABLE 1
Results of the half-side test

| | Type 1 | Type 2 | Type 3 | Type 4 |
|---|---|---|---|---|
| Cationic surfactant | | | | |
| according to formula | | | | |
| (I) where | | | | |
| R1 = | C17-alkyl | C17-alkyl | C17-alkyl | C17-alkyl |
| R2 = | CH₃ | CH₃ | CH₃CH₂ | CH₃ |
| R3 = | CH₃ | H | CH₃ | CH₃ |
| n = | 2 | 2 | 2 | 3 |
| X = | Cl | Cl | Cl | Cl |
| Skin feel | + | ++ | 0 | − |
| Wet combability | ++ | + | 0 | − |
| Feel of wet hair | ++ | ++ | 0 | − |
| Dry combability | + | ++ | 0 | −− |
| Feel of dry hair | + | ++ | 0 | −− |
| Curl retention | + | 0 | 0 | 0 |
| Shine | + | 0 | 0 | 0 |
| Volume | 0 | 0 | 0 | 0 |
| Anti-electrostatic | 0 | 0 | 0 | 0 |
| effect | | | | |
| Stylability | + | 0 | 0 | 0 |

The determinations were carried out against an identical formulation which comprised behentrimonium chloride instead of the cationic surfactant according to the present invention.

The following apply here:

−− significantly poorer than formulation with behentrimonium chloride
− poorer than formulation with behentrimonium chloride
0 comparable with formulation with behentrimonium chloride
+ better than formulation with behentrimonium chloride
++ significantly better than formulation with behentrimonium chloride

Table 2 impressively shows that the cationic surfactants type 1 and type 2 have the best properties and have better conditioning properties than behentrimonium chloride. Type 3 is at least comparable.

### Example 3

### Biodegradability and Toxicity

For the cationic surfactant type 1 from Table 2, the biodegradability and toxicity was determined in accordance with OECD 301 B against algae. Here, it was found that the product according to the invention is rapidly degraded and has a low toxicity of 50-100 mg/l towards algae.

In the same test, behentrimonium chloride is not degraded and has a considerably higher toxicity of <1 mg/l towards algae.

What is claimed is:

1. A cationic surfactant of formula (I): 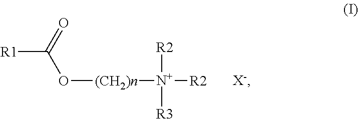
in which R1 is a saturated or unsaturated, branched or linear alkyl group having 6 to 22 carbon atoms, R2 and R3 independently are C1-C3 alkyl groups or hydrogen, n is 2 or 3, and X⁻ is an anion selected from the group consisting of halides, deprotonated carboxylic acids and methosulfate.

2. The cationic surfactant of claim 1, wherein R1 is a saturated or unsaturated, branched or linear alkyl group having 12 to 22 carbon atoms, R2 is methyl, R3 is methyl or hydrogen, n is 2, and X⁻ is chloride.

3. The cationic surfactant of claim 2, wherein R1 is a saturated or unsaturated, branched or linear alkyl group having 18 to 22 carbon atoms.

4. A cosmetic preparation comprising at least one cationic surfactant of claim 1.

5-6. (canceled)

7. A method for producing a cosmetic preparation, comprising adding at least one of the cationic surfactants of claim 1 to a cosmetic composition base.

8. A method of conditioning hair, comprising treating hair with a cosmetic preparation comprising at least one cationic surfactant of claim 1.

9. The cosmetic preparation of claim 4, wherein said cationic surfactant is present in an amount of 0.1% to 20% by weight, based on the preparation.

10. The method for producing a cosmetic preparation of claim 7, wherein said cationic surfactant is added in an amount of 0.1% to 20% by weight, based on the preparation.
